(19) United States
(12) Patent Application Publication
(10) Pub. No.: US 2018/0318356 A1
(43) Pub. Date: Nov. 8, 2018

(54) DERMATOLOGICAL AND COSMETIC TREATMENTS USING MESENCHYMAL STEM CELLS

(71) Applicants: Kenneth Allen Pettine, Ft. Collins, CO (US); Edward Jeffrey Donner, Loveland, CO (US)

(72) Inventors: Kenneth Allen Pettine, Ft. Collins, CO (US); Edward Jeffrey Donner, Loveland, CO (US)

(21) Appl. No.: 15/856,698

(22) Filed: Dec. 28, 2017

Related U.S. Application Data
(60) Provisional application No. 62/440,101, filed on Dec. 29, 2016.

(51) Int. Cl. A61K 35/28; A61K 8/73; A61K 8/98; A61Q 19/08; A61K 9/06

(52) U.S. Cl. CPC A61K 35/28; A61K 8/735; A61K 2035/124; A61Q 19/08; A61K 9/06; A61K 8/981

(57) ABSTRACT

The present disclosure provides inventive cosmetic/dermatological formulations and/or preparations that include mesenchymal stem cells (MSCs), and also provides associated methods for repairing, rejuvenating and/or augmenting skin tissue. The formulations and/or preparations include MSCs that are formulated into creams, lotions, and the like. In one aspect, the MSCs are obtained autogenously. In another aspect, the MSCs are obtained from allogeneic sources. In another aspect, the MSCs may be minimally manipulated. In another aspect, the MSCs may be derived from bone marrow or adipose tissue. In another aspect, the MSCs can be utilized from bone marrow concentrate (BMC) or from adipose stromal vascular fraction (SVF). In another aspect, the MSCs can also be utilized after cell expansion. The associated methods comprise topical application of the cosmetic/dermatological formulations and/or preparations onto skin tissue, resulting in repairing, rejuvenating, and/or augmenting the skin tissue.

### CROSS REFERENCE TO RELATED APPLICATIONS

This utility patent application claims the benefit of, and priority to U.S. provisional patent application Ser. No. 62/440,101, titled DERMATOLOGICAL AND COSMETIC TREATMENTS USING MESENCHYMAL STEM CELLS, filed on Dec. 29, 2016, the entire disclosure of which is incorporated herein by reference.

### FIELD OF THE INVENTION

The present disclosure relates to dermatological and/or cosmetic formulations and/or preparations, including creams, lotions, and the like, that comprise stem cells, which are useful for treating skin tissue. The disclosure also relates to methods of use of said dermatological and/or cosmetic formulations and/or preparations that comprise stem cells for treatment of skin tissue.

### BACKGROUND OF THE INVENTION

Skin is composed of the epidermis and the dermis. Below these layers lies the hypodermis, which is not usually classified as a layer of skin. The hypodermis is also commonly referred to as subcutaneous fat layer, or subcutaneous tissue. The outermost epidermis is made up of stratified squamous epithelium with an underlying basement membrane. It contains no blood vessels, and is nourished by diffusion from the dermis. The main type of cells which make up the epidermis are keratinocytes. Also present are melanocytes and Langerhans cells. This layer of skin is responsible for keeping water in the body and keeping harmful chemicals and pathogens out.

The dermis lies below the epidermis and contains several structures including blood vessels, nerves, hair follicles, smooth muscle, glands, and lymphatic tissue. The dermis (or corium) is typically 3-5 mm thick and is the major component of human skin. It is composed of a network of connective tissue, predominantly collagen fibrils providing support and elastic tissue providing flexibility. The main cell types are fibroblasts, adipocytes, and macrophages. The hypodermis lies below the dermis. Its purpose is to attach the skin to underlying bone and muscle as well as supplying it with blood vessels and nerves. It is made up of loose connective tissue and elastin. The main cell types are fibroblasts, macrophages, and adipocytes. The hypodermis contains 50% of body fat. Fat serves as padding and insulation for the body.

Skin is subject to several conditions that can lead to loss of elasticity, wrinkling and age spots, and a resultant desire for cosmetic enhancement. Such conditions include aging due to chronological aging or photoaging from exposure to the sun, or both. Skin aging results in wrinkling, the appearance of pigmented areas (“age spots”), thinning of the skin, loss of elasticity, and other undesirable characteristics. Other skin conditions, which can be improved by cosmetic approaches, include reduction in visible scarring, for example, from acne or other causes, uneven pigmentation, and the like.

A major cause of the deterioration of facial and other skin is aging. Facial aging occurs as the result of several factors: inherent changes within the skin, effects of gravity, facial muscles acting on the skin (dynamic lines), soft tissue loss or shift and bone loss and loss of tissue elasticity. The skin ages when the epidermis begins to thin, causing the junction with the dermis to flatten. Collagen decreases as a person ages and the bundles of collagen, which give the skin turgor, become looser and lose strength. When the skin loses elasticity, it is less able to resist stretching. Coupled with gravity, muscle pull and tissue changes, the skin begins to wrinkle. Water loss and breakdown of bonds between cells also reduces the barrier function of the skin, which can cause the skin's pore size to increase.

As a person ages, the face loses volume, soft tissue, and fat. The appearance of jowls and folds is usually caused by the drooping of facial tissues and folding of areas where the muscles below are attached to the skin. As part of the reduction in soft tissue the face gets more hollow.

More specifically, in various facial areas, such as forehead, eyes, nose, midface and lower face, changes relating to aging have been well documented. In the forehead area, the forehead and brow droop over time, which lowers the eyebrows and causes the upper eyelid skin to bunch. Forehead lines appear when one tries to hold the brows and eyelids up to counteract these changes. It is well known that the eyes are often the first facial feature to show signs of aging. Skin changes around the eyes occur earlier than in the rest of the face since the skin is thinner around the eyes. The skin here contains fewer glands and is subjected to constant blinking, squinting, rubbing, and pulling. The midface ages when the cheeks begin to droop, causing nasolabial folds. Nasolabial folds are the lines that run from the sides of the nose to the corners of the mouth. These folds have been treated with facial fillers. In the nose area, as a person ages, the nose elongates. Common causes of elongation are thinning of the soft tissue and loss of elasticity, which causes “drooping of the tip” and unmasking of the bone, creating a new hump. In the lower face area, as the face ages, facial tissues descend. This results in the so-called “laugh lines”. Folds and lines in this area have been treated with facial fillers. Further down on the face, the corners of the mouth may droop, and descent of the jowls can create folds often referred to as “marionette” lines. Furthermore, jowls form when the cheeks sag around a fixed point along the jaw where the facial muscles attach to the jawbone. The facial muscles continue down into the neck as a sheet called the platysma muscle. This muscle often gaps in the center of the neck, creating two bands.

A variety of procedures have been developed for improving skin appearance. Examples of such procedures include treatment with Botulinum Toxin Type A (“Botox”), retinoids and derivatives thereof and especially retinoic acid (all-trans and 13-cis) and retinol, and the use of hydroxy acids. Although progress has been made in the use of cosmetic compositions for the treatment of skin, response to treatment is variable and often a condition is only marginally to moderately responsive to treatment. Unfortunately, once applied to the skin, some of these agents can cause itching, stinging and tightness, which may lead to considerable discomfort, and a progressive immune reaction to the product. For many subjects, sensitivity to sun is enhanced. The use of these products by consumers with sensitive skin is often prevented. Many of the same considerations apply to other methods for cosmetic treatment of skin, such as laser resurfacing or dermabrasion. Thus, there remains a need for cosmetic and/or dermatological compositions and methods for the treatment of skin that does not cause immune reaction to product in subjects and that can be used by subjects with sensitive skin.

In recent years, some researchers have reported on skin treatments that use injectable compositions that include stem cells and a variety of additional components. For example, Giampapa, V., et al. (“Method and Composition for Restoration of Age-Related Tissue Loss in the Face or Selected Areas of the Body,” U.S. patent application Ser. No. 13/318,524) disclose an injectable composition and a method of its use to restore skin. The composition includes stem cells (e.g., peripheral blood stem cells, bone marrow-derived blood stem cells, or mesenchymal stem cells), hyaluronic acid as a carrier, growth factors, and adipocytes; and the method includes pretreatment of the skin by performing a microdermabrasion step prior to injection.

Also recently, some researchers have reported on the possible treatment of skin with stem cells by including the stem cells in compositions intended for topical application, thus providing a gentler alternative to injectable treatments. For example, in the above Giampapa patent application, it is stated that the stem cell composition may be applied topically to the skin. However, no data is presented to show that these researchers had actually used their composition on a subject topically and that benefits have been observed.

Schliefelbein, J. (“Cosmetic Preparation and Method to Obtain a Somatic Stem Cell Preparation,” European Patent Application No. EP2039348) discloses a cosmetic preparation containing somatic stem cells and a simple method to obtain a somatic stem cell preparation and its use, in particular to ameliorate the appearance of the skin. The stem cells are reportedly isolated from human sternum in a simple, fast, and painless way. It is stated that this method advantageously makes an ambulant cosmetic or therapeutic treatment with stem cells in or even outside a hospital possible. Alternatively, the stem cells are isolated from animal bone marrow and preferably applied to the skin in the form of a cream, salve, lotion, gel or wax.

Lee, S., et al. (“Mesenchymal Stem Cell-Hydrogel-Biodegradable or Mesenchymal Stem Cell-Hydrogel-Undegradable Support Composition for Skin Regeneration or Wound Healing,” US Patent Application Publication No. US20160051722) disclose a composition comprising mesenchymal stem cell-hydrogel-biodegradable support or mesenchymal stem cell-hydrogel-undegradable support, a sheet comprising the composition and a method for the preparation thereof. It is stated that, by using the sheet comprising the adipose-derived mesenchymal stem cell-hydrogel biodegradable or undegradable support, stem cells of high activity may be applied directly to a wound without isolation process using protease. It is also stated that the sheet has extracellular matrices such as collagen, laminin, fibronectin, and elastin secreted by stem cells in the culture stage and included completely in the hydrogel, and has superior skin regeneration and wound healing effects compared with conventional pharmaceutical preparations, and shortens the therapeutic period.

Crawford, K. D., et al. (“Compositions Having Early Lineage Adult Stem Cells and Extracts Thereof,” PCT Patent Application Publication No. WO2015017775) disclose various dermatological and cosmetic compositions for topical application to a subject that include early lineage adult stem cells (ELA cells) and extracts or formulations thereof, and claim that such compositions can effectively improve the health or appearance of a subject's skin. The ELA cells are apparently obtained from the synovial fluid of patients with osteoarthritis of the knee. However, it appears that the use of this technology has been the subject of serious questions by other researchers in the field, particularly regarding obtention of ELA cells from allogeneic sources. Additionally, efforts by some leading researchers in the field to re-create the ELA cells in order to test their reported efficacy and reproduce Crawford's data have been met with failure. Nonetheless, in a recent journal publication (Crawford, K. D., et al., “Isolation and Characterization of Early Lineage Adult Stem Cells from the Synovial Fluid of Osteoarthritis Patients,” J J Regener Med. 2015, 1(1): 005), Crawford provides detailed description of the isolation, characterization, and properties of the ELA cells. It is important to note that in WO2015017775, page 4, lines 12-17, it is stated: “ELA cells are distinct from embryonic stem cells (ESC) and other types of early lineage adult cells such as MSC, VSEL, MAPC and cord blood derived progenitor cells because the ELA stem cells do not appear to detectably express the various stem cell markers CD34, CD44, CD45, CD49a, CD66A, CD73, CD90, CD105, CSCR4, SSEA, or MHC class I or MHC class II structures, found on other cell types.” In contrast, mesenchymal stem cells (MSCs) are known as being positive for CD73, CD90, and CD105. Likewise, in the above journal publication, on page 2, column 1, first paragraph, it is stated: “MSCs most commonly express surface markers such as CD29, CD44, CD49a-f, CD51, CD73, CD105, . . . ” And, on page 5, column 1, last paragraph, it is stated regarding ELA cells: “Further analysis revealed no expression of CD73, CD90, CD105, . . . ” Also, on page 12, column 1, under Conclusion, it is stated: “ELA cells are also molecularly distinguishable from ESCs, IPS cells, and MSCs by their unique gene expression patterns.” Thus, evidently, based on the foregoing, ELA cells are quite different than MSCs.

All publications cited throughout this application are incorporated herein by reference in their entirety.

MSCs, after their initial discovery in bone marrow, have been isolated and characterized from several adult and fetal tissues, including adipose (fat), dermis (skin), synovial fluid, periosteum, umbilical cord blood, placenta, and amniotic fluid. MSCs are partially defined by their ability to differentiate into tissues including osteoblasts (bone cells), chondrocytes (cartilage cells), myocytes (muscle cells), and adipocytes (fat cells). But it is believed that it is their trophic, paracrine, and immunomodulatory functions that may have the greatest therapeutic impact in vivo. Unlike pharmaceutical treatments that deliver a single agent at a specific dose, MSCs are site regulated and secrete bioactive factors and signals at variable concentrations in response to local microenvironmental cues. Significant progress has been made in understanding the biochemical and metabolic mechanisms and feedback associated with MSC response. The anti-inflammatory and immunomodulatory capacity of MSC may be paramount in the restoration of localized or systemic conditions for normal healing and tissue regeneration. Allogeneic MSC treatments, categorized as a drug by regulatory agencies, have been widely pursued, but new studies demonstrate the efficacy of autologous MSC therapies, even for individuals affected by a disease state. Safety and regulatory concerns surrounding allogeneic cell preparations make autologous and minimally manipulated cell therapies an attractive option for many regenerative, anti-inflammatory, and autoimmune applications. (See, Murphy, M. B., et al., “Mesenchymal stem cells: environmentally responsive therapeutics for regenerative medicine,” Experimental & Molecular Medicine, 45:e54 (2013)).

The primary trophic property of MSCs is the secretion of growth factors and other chemokines to induce cell proliferation and angiogenesis. MSCs express mitogenic proteins such as transforming growth factor-alpha (TGF-α), TGF-β, hepatocyte growth factor (HGF), epithelial growth factor (EGF), basic fibroblast growth factor (FGF-2) and insulin-like growth factor-1 (IGF-1) to increase fibroblast, epithelial and endothelial cell division. Vascular endothelial growth factor (VEGF), IGF-1, EGF and angiopoietin-1 are released to recruit endothelial lineage cells and initiate vascularization. It has been hypothesized that an individual's genotype has a role in the expression of and reaction to these cytokines, providing credence to the philosophy of personalized medicine utilizing responsive agents (that is, MSCs) rather than a dose of recombinant proteins or autologous growth factors (for example, platelet-rich plasma). The trophic effects extend beyond cell proliferation to the reduction of scar tissue formation presumably by local cells secreting paracrine factors keratinocyte growth factor, stromal cell-derived factor-1 (SDF-1) and macrophage inflammatory protein-1 alpha and beta.

Regarding the anti-inflammatory and immunomodulatory properties of MSCs, it is believed that, in many types of musculoskeletal trauma, inflammatory conditions at the site of injury impede the natural repair processes by local progenitor and mature cells. Without being bound by theory, it is believed that MSCs assist via paracrine mechanisms and modulate the regenerative environment via anti-inflammatory and immunomodulatory mechanisms. In response to inflammatory molecules such as interleukin-1 (IL-1), IL-2, IL-12, tumor necrosis factor-α (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-β1, HGF, SDF-1, nitrous oxide, indoleamine 2,3-dioxygenase, IL-4, IL-6, IL-10, IL-1 receptor antagonist and soluble tumor necrosis factor-a receptor. MSCs prevent proliferation and function of many inflammatory immune cells, including T cells, natural killer cells, B cells, monocytes, macrophages, and dendritic cells. Although MSCs across species are able to regulate T-cell activity, the mechanisms are not identical across mammalian species.

A characteristic of chronically inflamed environments is a persistent imbalance in the types of helper T cells and macrophages. MSCs indirectly promote the transition of TH1 to TH2 cells by reducing INF-g and increasing IL-4 and IL-10. The restored TH1/TH2 balance has been shown to improve tissue regeneration in cartilage, muscle, and other soft tissue injuries, alleviate symptoms of autoimmune diseases and have an anti-diabetic effect. Similarly, reduction in INF-γ and secretion of IL-4 promotes a shift in macrophages from M1 (pro-inflammatory, anti-angiogenic and tissue growth inhibition) to M2 (anti-inflammatory, pro-remodeling and tissue healing) type, an effect required for skeletal, muscular, and neural healing and regeneration. (See, Murphy, M. B., et al., “Mesenchymal stem cells: environmentally responsive therapeutics for regenerative medicine,” Experimental & Molecular Medicine, 45:e54 (2013)).

Throughout this description, including the foregoing description of related art and cited publications, as well as any and all publications cited in what follows below, it is to be understood that any and all publicly available documents described herein, including any and all cited U.S. patents and patent applications, are specifically incorporated by reference herein in their entirety. Nonetheless, the related art and publications described herein are not intended in any way as an admission that any of the documents described therein, including pending U.S. patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE INVENTION

An embodiment of the disclosed invention provides formulations and methods for treating skin tissue using autogenous or allogeneic mesenchymal stem cells (MSC) derived from bone marrow or adipose tissue. In one aspect, the MSCs can be utilized from bone marrow concentrate (BMC) or from adipose stromal vascular fraction (SVF). In another aspect, the MSCs can also be utilized after cell expansion.

An embodiment of the invention provides a formulation including a cosmetic base having from about 0.01% to 10% w/w of an MSC preparation. In some embodiments, the MSC preparation is devitalized, extracted and or fractionated. In other embodiments, the MSC preparation is lyophilized. In some embodiments, the MSC preparation may further include a tissue growth factor. In other embodiments, the MSC preparation may further include a tissue growth factor and cell membrane fraction(s).

In some embodiments of the invention the MSC preparation includes expanded MSCs. Some embodiments of the invention further include from about 0.01% to 10% w/w of cell-free medium conditioned by growth of MSCs and/or MSC-lineage cells. In some embodiments, the media is lyophilized. In some embodiments, the invention is substantially free from non-human animal products.

Another embodiment of the invention provides use of MSCs, MSC lysates, and/or MSC products in the manufacture of a medicament. In an embodiment of the invention the use thereof results in improvement in the appearance or health of the skin of a subject.

Another embodiment of the invention provides a method of treating a skin condition including: wrinkling, folds, sagging, age spots, uneven pigmentation, thinning, elasticity, scarring, surface roughness, surface roughness, surface vessels, redness, and pore size, including administering to a subject having one or more of the aforesaid conditions, a dose of a formulation including, a cosmetic base having from about 0.01% to 10% w/w of an MSC preparation.

Another embodiment of the invention provides a method of skin tissue rejuvenation, augmentation, and/or repairing, including inducing tissue growth and recovery from standard cosmetic procedures by administering to a subject subdermally a preparation of 10² to 10⁶ live MSCs.

Another embodiment of the invention provides a method of skin tissue rejuvenation, augmentation, and/or repairing, including inducing tissue growth and recovery from standard cosmetic procedures by administering to a subject externally the formulation, including a cosmetic base having from about 0.01% to 10% w/w of MSCs, MSC cell extract, or cell-free media preparation.

Another embodiment of the invention provides a method of skin tissue rejuvenation, augmentation, and/or repairing, including inducing tissue growth and recovery from standard cosmetic procedures by administering to a subject subdermally, a preparation of 10² to 10⁶ live MSCs and subsequently administering externally one or more doses of the formulation, including a cosmetic base having from about 0.01% to 10% w/w of MSCs, MSC cell extract, or cell-free media preparation.

In some embodiments, the mode of administration is topical, and optionally, other cosmetics or devices, may be in contact on the surface or below the dermal surface at the same time. In some embodiments, the cosmetic base is a lotion. In other embodiments, the cosmetic base is a cream. In some embodiments, the cosmetic base includes a pigment. In another embodiment, the cosmetic base includes a sunscreen. In some embodiments, the cosmetic base includes at least one anti-acne agent. In other embodiments, the cosmetic base includes liposomes. In some embodiments, the cosmetic base includes at least one antioxidant. In other embodiments, the cosmetic base includes a platelet-rich fibrin matrix. An embodiment of the invention provides a composition, including a cosmetic base having from about 0.01% to 10% w/w of one or more of: an MSC preparation, MSC extract, and MSC-conditioned medium; the MSC-conditioned medium generated in an environmentally-closed cell culture system. In an embodiment of the invention the medium is filter-sterilized. In an embodiment of the invention the medium is concentrated.

It is to be understood that, as contemplated herein, any suitable biologic MSC product may be utilized in the various embodiments of the invention disclosed herein. In a preferred embodiment, the biologic MSC product is BMC. BMC is an excellent source of MCSs, because the MSCs are stored in bone marrow. Illustrative examples of other sources of MSCs are peripheral blood, synovium, periosteum, skeletal muscle, and adipose tissue, and any other source of MSCs known to those skilled in the art.

MSCs obtained from BMC have been reported to possess many positive attributes. MSCs are believed to be anti-inflammatory, secrete numerous growth factors, stimulate blood vessel formation, modulate the immune system to enhance healing, fight bacteria, turn into localized cells, and potentially heal inflammation. Accordingly, in one broad embodiment, disclosed herein is a method of treating a skin condition, wherein the skin condition is associated with inflammation in the skin tissue, resulting in reduction of the inflammation and in improvement and/or restoration of the skin tissue.

MSCs are the preferred cells for the purpose of this invention. MSCs have been shown to have the potential to differentiate into several lineages including bone (Haynesworth et al. (1992) 13 Bone 81-88), cartilage (Mackay et al. (1998) 4 Tissue Eng 41 5-28; Yoo et al. (1998) 80 J Bone Joint Surg Am 745-57), adipose tissue (Pittenger et al. (2000) 251 Curr Top Microbiol Immunol-11), tendon (Young et al. (1998) 16 J Orthop Res 406-13), muscle, and stroma (Caplan et al. (2001) 7 Trends Mol Med 259-64). Nonetheless, as contemplated herein, it should be understood that a variety of stem cells other than MSCs may be used.

In another embodiment of the invention, disclosed herein is a method of skin tissue rejuvenation, augmentation, and/or repairing with autogenous MSCs resulting in improvement and/or restoration of the skin tissue. In one aspect, this method does not require inclusion of commonly used cosmetic dermal fillers and carriers such as hyaluronic acid and the like. However, fillers and carriers may optionally be used if desired. In another aspect, this method does not include nor require the addition of growth factors such as insulin, insulin-like growth factor, a thyroid hormone, a fibroblast growth factor, an estrogen, retinoic acid, and the like. In yet another aspect, this method does not include nor require the addition of adipocytes. In a further aspect, this method does not include nor require the inclusion of a step of preparing the skin by microdermabrasion or similar skin therapy to promote cellular repair. Nonetheless, as contemplated herein, it is understood that there is nothing in the disclosed invention that would prevent the use in the invention of any of the additives or steps listed in this paragraph, if desired.

In another embodiment of the invention, disclosed herein is a method of treatment of skin tissue with autogenous MSCs resulting in rejuvenation, augmentation, and/or repairing of the skin tissue. In one aspect, this method does not include nor require inclusion of a step of culturing and/or culture-expanding the MSCs. Nonetheless, optionally a step of culturing and/or culture-expanding the MSCs may be included.

In another embodiment of the invention, disclosed herein is a method of treating skin tissue in one or more areas of the body with autogenous MSCs, resulting in rejuvenation, augmentation, and/or repairing of the skin tissue. Illustratively, the one or more areas that may be treated include, but are not limited to, one or more areas of the face such as the peri-orbital area, the lips, the malar area, the nasolabial folds, and the labio-mandibular folds, and other areas of the body such as the neck and the hands. However, it is understood that skin tissue in other areas of the body may be treated in likewise manner.

In another embodiment of the invention, disclosed herein is a method of treating skin tissue of burn victims with autogenous MSCs, wherein the skin tissue has been scarred with the burns, resulting in healing and regrowing of the skin tissue.

Naturally, further objects of the invention are disclosed throughout other areas of the specification, drawings, and claims.

### BRIEF DESCRIPTION OF THE DRAWINGS

FIG. 1 is an illustration of the pelvic girdle showing a needle advanced through the iliac crest and into bone marrow to aspirate bone marrow from the ilium.

### DETAILED DESCRIPTION OF THE INVENTION

Before the present methods, implementations and systems are disclosed and described, it is to be understood that this invention is not limited to specific components, specific methods, specific implementation, or to particular compositions, and as such may, of course, vary. It is also to be understood that the terminology used herein is for the purpose of describing particular implementations only and is not intended to be limiting. Neither are mechanisms which have been provided to assist in understanding the disclosure meant to be limiting.

The present invention includes skin tissue rejuvenation, augmentation, and/or repairing compositions that are useful in providing improvements to the skin health and/or appearance of a human subject, including but not limited to improvements with respect to wrinkling, folds, sagging, age spots, uneven pigmentation, thinning, elasticity, scarring, surface roughness, surface vessels, redness, and pore size; and also in helping regrow the skin of burn victims. According to one embodiment, such a composition is achieved by incorporating a population of fresh or expanded MSCs, or extracts or derivatives thereof. According to another embodiment, such a composition is achieved by incorporating BMC that includes a population of fresh or expanded MSCs.

For the purposes of this disclosure, MSCs may be derived from multiple sources including, bone marrow stroma, adipose, blood, dermis, periosteum and tissues. In various embodiments, MSCs are extracted from patient intended to receive the cellular therapy (i.e., an autologous transplant). MSCs can be culture expanded prior to their introduction into the patient. Growth of MSCs in vitro can be used, for example, to increase the number of MSCs available for implantation or injection. In non-limiting examples, MSC numbers are increased about two-fold or greater, about ten-fold or greater, or about twenty-fold or greater or more, depending on the desired number of cells. Patient-specific master cell banks, for example can include ten or more passages of expanded autologous cells, which increases MSC counts by many orders of magnitude in comparison to primary isolates. In various embodiments, growing MSCs in vitro can include expansion in a cell culture medium, which includes nutrients, buffers, salts, proteins, vitamins and/or growth factors, which promote MSC growth. A useful cell culture medium is RPMI 1680 supplemented with 10% serum, and appropriate antibiotics such as penicillin/streptomycin and G418. Human serum is preferred for expanding the MSCs for human transplant preparations, but fetal bovine serum has produced acceptable yields of the MSCs in culture. A currently preferred culture system is an environmentally controlled, closed-culture system, commonly known as a bioreactor, which is particularly useful for collection of MSC-conditioned media. Other systems are suitable for culture of MSCs, the expansion of donor cells, and the creation of master MSC banks. These should comply with Good Tissue Practices and ideally should be cGMP. The expanded cells are commonly but not necessarily frozen prior to implantation, and a suitable cryogenic medium that is acceptable to the FDA for such purposes is CryoStorCS1O, available from BioLife Solutions, Bothell, Wash.

In one embodiment, the cosmetic composition having MSCs is substantially free from non-human animal products. Another embodiment of the invention provides for the use of a medium conditioned by growth of MSCs to prepare a medicament or cosmetic for administration to skin of a subject as a cellular rejuvenation compound. Another embodiment of the invention provides for the use of lysates from devitalized MSCs to prepare a medicament for a cosmetic or for the administration to a subject as a cellular rejuvenation compound. Another embodiment of the invention provides for the use of MSC-derived products from primary, expanded or transgene expressing MSCs to prepare a medicament for the administration to a subject as a cellular rejuvenation compound.

The present invention provides various dermatological and cosmetic compositions for topical application to an individual that includes MSCs, MSC lysates, and MSC products, each alone or in combination.

According to one embodiment of the invention, the composition is formulated for application to the skin of an individual. The amount of MSCs in this dermatological or cosmetic composition is an amount capable of providing a detectable improvement in the health or appearance of an individual's skin compared to untreated skin of the individual. Generally, a 0.01% to 10% w/w of cell extract, cell conditioned media or lyophilized MSCs, applied over a 30 to 60 day period, results in detectable improvement. In certain embodiments, the compositions of the invention include lotions, creams, oils, gels, including hydrogels, powders, serums, salves, foundations, facial masks, lip care products, sunscreens, hair care products, such as shampoos, conditioners, including deep conditioners, hair care treatments, hot oil treatments, skin cleansers, exfoliants, ointments, compact formulations, or the like.

The term cosmeceutical refers to a formulation or composition including at least one biologically active ingredient that has an effect on the user of the product and at least one cosmeceutically-acceptable carrier. Cosmeceuticals may be viewed as cosmetics that, in certain applications and under appropriate conditions, provide medicinal or drug-like benefits. In certain applications, for example, cosmeceuticals affect the underlying structure of the skin, decrease wrinkle depth, or reverse or ameliorate the effect of photooxidation or aging on the skin. Cosmeceuticals are particularly useful as skin care products, hair care products, and sun care products. In certain embodiments, cosmeceutical compositions include delivery systems including at least one of liposomes, cyclodextrins, polymer systems, or hyaluronic acid or related compounds. Cosmeceutical compositions include cosmeceutically-acceptable carriers.

A topical cosmetic or cosmeceutical ointment, lotion, or gel composition typically contains an effective amount of MSCs, and cell products derived therefrom. The cosmeceutical ointment may also include other active and inert ingredients in a cosmetically- or a cosmeceutically-acceptable carrier, such as a pharmaceutical cream base, an oil-in-water emulsion, a water-in-oil emulsion, or a gel. Various cosmetic and cosmeceutical compositions for topical use include drops, tinctures, lotions, creams, salves, serums, solutions, and ointments containing conditioned media or extracts, and an appropriate carrier. The optimal percentage of the conditioned media or extract in each composition varies according to the composition's formulation and the therapeutic effect desired. The skilled artisan in the formulation arts will understand that the inventive compositions may include any of a number of cosmetically-, cosmeceutically-, or pharmaceutically-acceptable formulations, depending on the type of product, the nature of the composition, the location of composition's use, the desired effect, and the like. While proprietary formulations are common in the formulation arts, formulators of ordinary skill will be able to determine or readily select appropriate formulations for specific applications without undue experimentation.

Discussions of cosmetic- and cosmeceutically-acceptable ingredients and formulations are found in the FDA Cosmetics Handbook, U.S. Food and Drug Administration; Handbook of Cosmetic and Personal Care Additives, Ash and Ash, compilers, 1994, Chemical Publishing, New York, N.Y.; Bennett's Cosmetic Formulary, 1993, Chemical Publishing Co.; Harry's Cosmeticology, 7.sup.th ed.5 Wilkinson & Moore, 1982 and δ.sup.th ed., Rieger, 2000, Chemical Publishing; Cosmetic Bench Reference-2001, Allerud Publishing Corp.; CTFA Compendium of Cosmetic Ingredient Composition, Nikitakis and McEwen, eds., 1990, Cosmetic, Toiletry, and Fragrance Association, Washington, D.C., Surfactant Encyclopedia, 2.sup.nd revised edition, Rieger, 1996, Allured Publishing; The Chemistry and Manufacture of Cosmetics, 2.sup.nd ed., De Navarre, Van Nostrand, Princeton, N.J.; Encyclopedia of Common Natural Ingredients Used in Food, Drugs, and Cosmetics, Leung, 1996, John Wiley; A Consumer's Dictionary of Cosmetic Ingredients, 5.sup.th ed., Winter, 1999, Three Rivers Press, New York, N.Y.; Cosmeceuticals: Active Skin Treatment, 1998, Allured Publishing; Handbook of Cosmetic Science and Technology, Knowlton and Pearce, 1993, Elsevier Advanced Technology, Oxford, UK; Personal-Care Formulas, 1997, Allured Publishing; Beginning Cosmetic Chemistry, Scheuller and Romanowski, 1999, Allured Publishing; and Skin Permeation: Fundamentals and Application, Zatz, 1993, Allured Publishing. Discussions of pharmaceutically-acceptable ingredients and formulations may be found in, among other places, Remington's Pharmaceutical Sciences, 18.sup.th ed., Gennaro, ed., 1990, Mack Publishing.

The methods of the invention have wide applicability to cosmetic conditions. The mode of administration for cosmetic applications is typically topical, but administration and dosage regimens vary depending on the cosmetic condition for which modulation is sought.

The present invention provides methods, compositions, and kits for cosmetic use with individuals. The term “individual” as used herein includes humans as well as other mammals. In some embodiments, the compositions, methods, and/or kits are used to provide a cosmetic treatment to an individual desiring and/or in need of cosmetic treatment (e.g., young children subject to burn or other scarring may not desire treatment but may nonetheless be in need of treatment). The term “treating” or “treatment” as used herein includes achieving a cosmetic benefit. By cosmetic benefit is meant any desired modulation of the cosmetic condition being treated. For example, in an individual with wrinkling, cosmetic benefit includes eradication or lessening of the appearance of wrinkling. Also, a cosmetic benefit is achieved with the eradication or amelioration of one or more of the psychological symptoms associated with the underlying condition such that an improvement is observed in the patient, notwithstanding the fact that the patient may still be affected by the cosmetic condition. For example, MSCs provide cosmetic benefit not only when a cosmetic defect is eradicated, but also when an improvement is observed in the individual with respect to the cosmetic defect and its attendant consequences, such as psychological consequences. In some cases, methods and compositions of the invention may be directed at achieving a prophylactic benefit. A “prophylactic,” or “preventive” effect includes prevention of a condition, retarding the progress of a condition (e.g., skin aging), or decreasing the likelihood of occurrence of a condition. As used herein, “treating” or “treatment” includes prophylaxis.

As used herein, the term “effective amount” encompasses an amount sufficient to effect beneficial or desired cosmetic results. An effective amount is administered in one or more administrations. In terms of cosmetic treatment, an “effective amount” of MSCs is an amount that is sufficient to palliate, ameliorate, stabilize, reverse or slow the progression of a cosmetic condition, or to provide a desired effect such as cosmetic augmentation of a soft tissue. An “effective amount” may be of MSCs and MSC products or a combination thereof used alone or in conjunction with one or more agents used to modulate a cosmetic condition.

The skin is subject to a number of cosmetic conditions that result in alterations of function and/or appearance that are considered undesirable, and the manifestation of these conditions can lead to psychological discomfort or harm. In some cases, no defect may be present; however, it may be desirable to the subject to augment or alter the skin in such a way as to produce a cosmetically pleasing effect. Examples of cosmetic conditions that may be modulated by the present invention include, but are not limited to, skin aging, cosmetic defect, undesired pigmentation, and post-cosmetic procedure damage.

Skin aging includes chronological aging as well as photoaging, and may appear as wrinkling, lack of elasticity (for example sagging), uneven pigmentation, thinning of the skin and/or collagen so that veins and other underlying structures become more prominent, and the like. Skin aging is a major example of a skin condition that involves a decrease in cell proliferation and in cell function. As used herein, “skin aging” refers to alterations in the appearance and function of skin that occur with aging, such as wrinkling, loss of elasticity, sagging, uneven pigmentation (for example, “age spots” or “liver spots”), and loss of underlying tissue mass. Such conditions may be accelerated and/or exacerbated by exposure to ultraviolet radiation (“photoaging”) and other environmental conditions.

With age and/or exposure to UV radiation, the epidermis thins and the skin appendages atrophy. Hair becomes sparse and sebaceous secretions decrease, with consequent susceptibility to dryness, chapping, and fissuring. The dermis diminishes with loss of elastic and collagen fibers. Moreover, keratinocyte proliferation (which correlates with skin thickness and skin proliferative capacity) decreases with age.

An increase in keratinocyte proliferation and collagen production is believed to counteract skin aging, i.e., wrinkles, thickness, elasticity and repair. According to the present invention, MSCs can be used cosmetically to counteract, at least for a time, the effects of aging on skin. A formulation containing MSCs or MSC products, or a combination thereof may be applied topically in areas where it is desired to counteract skin aging.

Also included in the skin conditions that may be treated by the methods of the invention are cosmetic defects that, while not pathological or physiologically harmful, may nonetheless cause psychological distress, in some cases to the extreme. In these cases, it is desirable to correct a particular feature or features causing distress or, alternatively, enhance a feature considered desirable. In addition to skin aging, such conditions include, e.g., striae gravidarum and striae distensiae (“stretch marks), atrophic scarring (e.g., acne scarring), wound (e.g., traumatic wounds, chronic wounds, or burn wounds) or surgical scarring, thickened and cracked skin (especially on the feet), and hair loss. In the latter embodiments, the invention relates to the use of preparations that comprise MSCs or MSC products or combination thereof to enhance hair growth. Cells from which the hair is produced grow in the bulb of the follicle. They are extruded in the form of fibers as the cells proliferate in the follicle. Hair “growth” refers to the formation and elongation of the hair fiber by the dividing cells. In some embodiments, the methods of the invention provide a means for altering the dynamics of the hair growth cycle to induce proliferation of hair follicle cells, particularly stem cells of the hair follicle. The subject compositions and method can be used to increase hair follicle size and the rate of hair growth in individuals, such as humans, e.g., by promoting proliferation of hair follicle stem cells. In one embodiment, the method comprises administering to the skin in the area in which hair growth is desired an amount of MSCs, MSC products, or combination thereof sufficient to increase hair follicle size and/or the rate of hair growth in the animal, e.g., human. Typically, the composition is administered topically as a cream or lotion, and is applied on a daily basis until hair growth is observed and for a time thereafter sufficient to maintain the desired amount of hair growth.

Undesired pigmentation includes pigmentation over an area of the body that is different than the pigmentation desired by the individual. Undesired pigmentation can be the result of, e.g., photoaging, reaction to inflammation, or reaction to trauma such as surgical or accidental skin breakage, and the like. Undesired pigmentation includes altered or undesired pigmentation over small areas such as freckles, as well as altered or undesired pigmentation over larger areas, such as, for example, uneven pigmentation or larger areas of undesired pigmentation.

Further cosmetic uses of the methods of the invention include tissue augmentation through, generally, topical application, such as for lip enhancement. By “augment” is meant to include giving the appearance of greater fullness, generally through an increase in the tissue of the skin or underlying tissue. Any suitable skin area may be selected for augmentation by the methods of the invention.

In addition, the methods of the invention may be employed to enhance and/or accelerate recovery from standard cosmetic procedures, which are damaging to skin and/or underlying tissues. In addition, these procedures may be associated with lengthy recovery time and suboptimal results. Such procedures include chemical peel, dermabrasion, laser resurfacing, ablative resurfacing, non-ablative resurfacing, photodynamic therapy, noncoherent light phototherapy, breast lift, face lift, eyelid lift, forehead lift, neck lift, thigh lift, buttock lift, tummy tuck, and scar revision. As will be apparent to those of skill in the art, some of these procedures can require further skin firming (e.g., “lifting” procedures) while others are more extensively damaging to the surface of the skin and require assistance for healing in a timely and optimal fashion (e.g., chemical peel, dermabrasion, ablative and non-ablative skin resurfacing). In some embodiments, the methods of the invention provide a method for achieving firming and lifting of the eyelids; this may be done either in place of or in conjunction with a conventional eyelid lift procedure. The methods of the invention may be used in conjunction with both types of procedures to enhance and/or accelerate healing and recovery. MSCs, MSC lysates, MSC products, or combinations thereof may be administered in any cosmetically acceptable carrier, as described in more detail below. The MSCs, MSC lysates, MSC products may be included in BMC, or adipose-derived stromal vascular fraction (SVF) In embodiments of methods of the invention, the concentration of MSCs, MSC lysates derived therefrom, and MSC products derived therefrom, or a combination thereof used may be more than about 0.00001, 0.00005, 0.0001, 0.001, 0.01, 0.1, 1, 5, 10 or 20% on a w/w basis. In some embodiments, the concentration of MSCs, MSC lysates, and MSC products or combination thereof is more than about 0.00005%. The concentration of MSCs, MSC lysates, and MSC products or combination thereof may be less than about 0.0001, 0.001, 0.001, 0.01, 0.1, 1, 5, 10, or 20% (all concentration percentages given herein are w/w unless otherwise indicated). In some embodiments, the MSCs, MSC lysates, and MSC products or a combination thereof are used at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations are contemplated. Skin coverage may also be described in terms of total μL of MSCs, MSC lysates, and MSC products or combination thereof/cm² of skin; in these terms, a typical coverage per administration would be more than about 5, 10, 100, 1000, 10,000, 50,000, or 500,000 μL/cm² of skin; less than about 1,000,000, 500,000, 50,000, 5000, 500, 50, or 5 μL/cm² of skin; or about 5 μL/cm² to about 500 μL/cm² of skin; or about 50 μL/cm² of skin.

The methods of the invention typically utilize topical administration, which may be by any suitable means that brings the MSCs, MSC lysates, and MSC products or combination thereof and, optionally, other cosmetic or dermatological agents, in contact with the surface of the skin. This would include application as a gel, lotion, cream, or liposomal preparation with or without occlusion, or application as a plaster, patch, mask, glove, or similar device for extended contact with an affected area of skin. For modulation of cosmetic conditions or to produce a desired cosmetic effect, the frequency and duration of administration of a formulation comprising a MSCs, MSC lysates, and MSC products or combination thereof is dependent on factors including the nature of the formulation (e.g., concentration, presence or lack of other cosmetic or dermatological agents, vehicle type), the severity and extent of the condition, and in some cases the judgment of a skin care professional, e.g., a health care professional such as a dermatologist, or a cosmetologist.

Topical application may be applied more than once, twice, three times, four times, five times, or six times per week, or more than once, twice, three times, four times, five times, or six times per day. Frequency of application may be less than about twice, three times, four times, five times, or six times per week, or less man about once, twice, three times, four times, five times, or six times per day. Some embodiments of the invention provide a method for cosmetic treatment of the skin of an individual by topical administration of an effective amount of MSCs, MSC lysates, and MSC products therefrom or a combination thereof. In some embodiments, the formulation is administered an average of about once per day; in some embodiments, the formulation is administered an average of about once or twice per day; in some embodiments, the formulation is administered an average of about one to three times per day; in some embodiments, the formulation is administered an average of more than about three times per day. In one embodiment, the formulation is administered an average of about twice per day, typically in the morning upon rising and in the evening before retiring. Topical administration may be without a covering. Alternatively, topical administration may include the use of a covering over the formulation, which may be occlusive or non-occlusive. For example, administration in the evening before retiring may include covering the administered area with an occlusive or non-occlusive covering, which may remain in place during sleep.

The duration of treatment generally will depend on the response of the skin to cosmetic treatment. Treatment may continue at the discretion of the individual being treated. In some cases, administration or application of a formulation containing MSCs, MSC lysates, and MSC products or combination thereof may be more frequent at the beginning of treatment and less frequent as treatment continues and the condition is ameliorated or the desired effect is achieved. In some cases, treatment may continue indefinitely in order to maintain a condition in abeyance or in an improved state, to delay onset of a cosmetic condition (e.g., skin aging), or to slow the progression of a cosmetic condition. These modifications of frequency and duration are easily accomplished by the individual being treated.

Some embodiments of cosmetic treatment of skin employ topical administration of a lotion, in some embodiments a mixture of emulsifying lanolin alcohols, waxes, and oils (e.g., EUCERIN™) or a mixture of petrolatum or mineral oil, a quaternary ammonium compound, a fatty alcohol, and a fatty ester emollient (e.g., CUREL, as described below, comprising MSCs, MSC lysates, and MSC products or combination thereof. The lotion containing the MSCs, MSC lysates, and MSC products or combination thereof is applied at a frequency of once to three times per day, in some embodiments once per day, until the desired result, e.g., reduction or elimination of wrinkling, sagging, and the like, is observed, followed by topical application once to three times per week, in some embodiments once per week, thereafter.

Some embodiments of cosmetic treatment of skin employ topical administration of a cream, in some embodiments a mixture of emulsifying lanolin alcohols, Water/Aqua/Eau, Petrolatum, Glycerin, Isostearyl Palmitate, Butylene Glycol, or Glyceryl Stearate (e.g., Creme LuXury™) or a mixture of, comprising of MSCs, MSC lysates, and MSC products or combination thereof. This composition is applied at a frequency of once to three times per day, in some embodiments once per day, until the desired result, e.g., reduction or elimination of wrinkling, sagging, and the like, is observed, followed by topical application once to three times per week, in some embodiments once per week, thereafter.

Some embodiments involve the cosmetic treatment of the eyelashes by employing a mascara in some embodiments a mixture of mineral oils, linseed oil, castor, eucalyptus, lanolin, or beeswax (e.g. COVERGIRL® NatureLuxe Mascara) or a mixture of, comprising of MSCs, MSC lysates, and MSC products or combination thereof. This composition is applied at a frequency of once to three-times per day, in some embodiments once per day, until the desired result, e.g. lengthening, thicken and/or curling of the eyelashes. In some instances, mascara may contain nanofibers, nanotubals, MSCs, MSC lysates, and MSC products or combination thereof to further lengthen, thicken, and/or curl the eyelashes.

The skilled artisan will understand that the appropriate carriers of the inventive compositions typically may include other ingredients typically found in the cosmetic and cosmeceutical fields: oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. The amounts of these various ingredients will vary depending on the use of the composition and the cosmetic or cosmeceutical effect desired.

If the MSCs, MSC lysates, and MSC products or combination thereof is used in combination with another skin care method or composition, any suitable combination of the MSCs, MSC lysates, and MSC products or combination thereof and the additional method or composition may be used. Thus, for example, if use of a MSCs, MSC lysates, and MSC products or combination thereof is in combination with another cosmetic or dermatological agent, the two may be administered simultaneously, consecutively, in overlapping durations, in similar, the same, or different frequencies, etc. In some cases, a composition will be used that contains a MSCs, MSC lysates, and MSC products or combination thereof in combination with one or more other cosmetic or dermatological agents.

Other dermatological or cosmetic agents that may be used in methods of the invention are described in more detail below. Dosages, routes of administration, administration regimes, and the like for these agents are well-known in the art.

As the cosmetic compositions are for topical use, they need not be sterile; however, if sterility is desired, it may be readily accomplished by methods known in the art, such as by filtration through sterile filtration (0.22 micron) membranes, or by other art-accepted means.

If desired, further cosmetic or dermatological ingredients may be incorporated in the formulations. The nature of the other ingredient(s) will depend on the cosmetic condition to be modulated and/or cosmetic result desired. These are described more fully below.

The MSCs, MSC lysates, and MSC products or combination thereof may be used neat (e.g., with an occlusive dressing so that the MSCs, MSC lysates, and MSC products or combination thereof is dissolved or dispersed in perspiration at the site), but generally is prepared in a vehicle suitable for topical administration. Compositions of the invention include MSCs, MSC lysates, and MSC products or combination thereof in a vehicle suitable for topical administration.

Numerous vehicles for topical application of cosmetic compositions are known in the art. See, e.g., Remington's Pharmaceutical Sciences, Gennaro, A R5 ed., 20.sup.th edition, 2000: Williams and Wilkins PA, USA. All compositions usually employed for topically administering cosmetic compositions may be used, e.g., creams, lotions, gels, dressings, shampoos, tinctures, pastes, ointments, salves, powders, liquid or semi-liquid formulation, patches, liposomal preparations, and the like. Application of said compositions may, if appropriate, be by aerosol e.g. with a propellent such as nitrogen carbon dioxide, a freon, or without a propellent such as a pump spray, drops, lotions, or a semisolid such as a thickened composition which can be applied by a swab. In particular compositions, semisolid compositions such as salves, creams, lotions, pastes, gels, ointments and the like will conveniently be used. The MSCs, MSC lysates, and MSC products or combination thereof may optionally be dissolved or diluted in a small amount of an appropriate solvent, such as ethanol or DMSO, before dispersion in the vehicle; however, this is not required. Compositions known in the art, preferably hypoallergenic and pH-controlled are especially preferred for topical administration, and include toilet waters, packs, lotions, skin milks or milky lotions. The preparations contain, besides the MSCs, MSC lysates, and MSC products or combination thereof and, optionally, other active ingredients, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like.

Examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetyl sulfate, polyoxyethylene lauryl ether phosphate, sodium N-acyl glutamate; cationic surfactants such as stearyldimethylbenzylammonium chloride and stearyltrimethylarnmonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleyl ether, polyethylene glycol monostearate, polyoxyethylene sorbitan monopalmitate, polyoxyethylene coconut fatty acid monoethanolamide, polyoxypropylene glycol (e.g. the materials sold under the trademark “Plutonic”), polyoxyemylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

For preparing compositions for topical administration, the concentration of MSCs, MSC lysates, and MSC products or combination thereof may be more than about 0.00001, 0.00005, 0.0001, 0.001, 0.01, 0.1, 1, 5 or 10% by weight. In some embodiments, the concentration of the MSCs, MSC lysates, and MSC products or combination thereof is more than about 0.00005%. The concentration of MSCs, MSC lysates, and MSC products or combination thereof may be less than about 0.0001, 0.001, 0.001, 0.01, 0.1, 1, 5, 10 or 20% (all concentration percentages given herein are w/w unless otherwise indicated). In some embodiments, lower and higher concentrations are contemplated. In some embodiments employing ointments, lotions, or creams, the pharmaceutically acceptable “base” is a carrier for example, mat can contain 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In water, the carrier for example consists of 2 to 10% of a lower alcohol, 0.1 to 10% or in particular 0.5 to 1% of a surfactant, 1 to 20%, in particular 3 to 7% of a humectant, 0 to S % of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume.

In some embodiments, MSCs, MSC lysates, and MSC products or combinations thereof, optionally with other active ingredients, are dissolved, mixed, or suspended in a mixture of emulsifying lanolin alcohols, waxes, and oils (e.g., EUCERIN™ Lotion) or a mixture of petrolatum or mineral oil, a quaternary ammonium compound, a fatty alcohol, and a fatty ester emollient (e.g., CUREL™ Lotion), or lotions that are substantially similar in composition. EUCERIN™ Dry Skin Therapy Original Moisturizing Lotion comprises water, mineral oil, isopropyl myristate, PEG-40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone. CUREL™ Fragrance Free Daily Moisturizing Lotion comprises water, glycerin, distearyldimonium chloride, petrolatum, isopropyl palmitate, cetyl alcohol, dimethicone, sodium chloride, methylparaben, and propylparaben.

Some embodiments of compositions of the invention comprise MSCs, MSC lysates, and MSC products or combination thereof in a lotion comprising a mixture of emulsifying lanolin alcohols, waxes, and oils (e.g., EUCERIN™ Dry Skin Therapy Original Moisturizing Lotion) at a concentration greater than about 0.00005%. Some embodiments of compositions of the invention comprise MSCs, MSC lysates, and MSC products or combinations thereof in a lotion comprising a mixture of emulsifying lanolin alcohols, waxes, and oils (e.g., EUCERIN™ Dry Skin Therapy Original Moisturizing Lotion) at a concentration of about 0.0001% to about 0.01%.

Some embodiments of compositions of the invention comprise MSCs, MSC lysates, and MSC products or combinations thereof in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration greater than about 0.00005%. Some embodiments of compositions of the invention comprise MSCs, MSC lysates, and MSC products or combination thereof in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.0001% to about 0.01%.

In addition, MSCs, MSC lysates, and MSC products or combinations thereof and, optionally, other active ingredients, may be formulated in liposome-containing compositions. Liposomes are artificial vesicles formed by amphipathic molecules such as polar lipids, for example, phosphatidyl cholines, ethanolamines and serines, sphingomyelins, cardiolipins, plasmalogens, phosphatide acids and cerebrosides. Liposomes are formed when suitable amphipathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers. The incorporation of MSCs, MSC lysates, and MSC products or combination thereof into liposomal preparations suitable for topical application can be achieved by a number of methods. With respect to liposomal preparations, any known methods for preparing liposomes for treatment of a condition may be used. See, for example, Bangham et al., J. MoL Biol, 23: 238-2S2 (1965) and Szoka et al., Proc. Natl Acad. Sci. 75: 4194-4198 (1978). Ligands may also be attached to the liposomes to direct these compositions to particular sites of action.

Liposomes containing MSCs, MSC lysates, and MSC products or combination thereof and, optionally, other ingredients can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methyl cellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. Exemplary metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose. The organic component consists of a suitable non-toxic, cosmetically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatidylserine, phosphatidylethanolamine. phosphatidylinositol, lysophosphatidylcholine and phosphatidyl glycerol, for example. Other lipophilic additives may be employed in order to selectively modify the characteristics of the liposomes. Examples of such other additives include stearylamine, phosphatide acid, tocopherol, cholesterol and lanolin extracts. In addition, other ingredients which can prevent oxidation of the phospholipids may be added to the organic component Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Although MSCs, MSC lysates, and MSC products or combinations thereof are generally capable of penetrating cell membranes and reaching the deep layers of skin, it may be useful in some embodiments to also include a penetration enhancer in the formulations of the invention. A penetration enhancer is a substance that improves cutaneous penetration of a bioactive substance. Suitable penetration enhancers include, for example, dimethyl sulfoxide (DMSO), DMSO-like compounds, ethanolic compounds, pyroglutamic acid esters and other solvents or compounds known to those skilled in the pharmaceutical art which facilitate dermal penetration of the drugs or chemicals chosen for the pharmaceutical composition. Various iontophoresis techniques can also be utilized to enhance penetration. Other penetration enhancers include amphiphiles such as L-amino acids, anionic surfactants, cationic surfactants, amphoteric surfactants, nonionic surfactants, fatty acids and alcohols. Additional penetration enhancers are disclosed in Remington: The Science and Practice of Pharmacy, 19th Edition (1995) on page 1583. The penetration enhancer chosen and the relative proportion of the penetration enhancer with respect to the active drugs or chemicals depends on the desired rate of delivery of the drugs or chemicals into the skin, which in turn depends on the condition being treated and the outcome sought More specifically, the type and amount of enhancer is chosen so that a sufficiently high concentration of active drugs or chemicals is attained in the skin to treat the condition within the time period considered desirable. Apart from the above-described compositions, use may be made of covers, e.g. plasters, bandages, dressings, gauze pads, patches and the like, containing an appropriate amount of MSCs, MSC lysates, and MSC products or a combination thereof and, optionally, other ingredients. In some cases use may be made of plasters, bandages, dressings, gauze pads, patches and the like which have been impregnated with a topical formulation containing the therapeutic formulation. Additional cosmetic and dermatological agents. Other cosmetic or dermatological agents may be included in methods and formulations of the invention. A “cosmetic or dermatological agent” as used herein, includes any substance whose administration for treatment of a cosmetic condition or to achieve a desired cosmetic effect, results in an status of the condition that is better than the status that would exist without the use of the cosmetic or dermatological agent

Anti-wrinkling agents are one form of cosmetic or dermatological agents. Anti-skin wrinkling agents include a variety of agents, often in combination, that prevent or treat wrinkling through a variety of actions. Many approaches are taken to reduce the appearance of facial and other wrinkles based on the understanding of the molecular basis of wrinkle formation. Such treatments include cosmetic products, drug therapy and surgical procedures. For example, many cosmetic products contain hydroxy acids, which may stimulate collagen synthesis. Another common treatment utilizes retinol, retinoic acid, retinol palmitate, a derivative of vitamin A, (or prescribed versions, Retin-A and Renova) which may directly or indirectly stimulate collagen production or retard collagen degradation. Bicyclic aromatic compounds with retinoid-type activity, which are useful in particular in preventing or treating various keratinization disorders, are described in EP 679630. These compounds are particularly active for repairing or combating chronological or actinic aging of the skin, for example such as in anti-wrinkle products. Antioxidants such as vitamin C and E and coenzyme Q-10 are believed to counteract free radicals, which damage cells and cause aging and have been used in treatments of wrinkles. Recently, the FDA approved cosmetic use of Botox (an extremely purified form of botulinum toxin) to treat glabella frown lines.

Thus, cosmetic or dermatological agents that complement cosmetic treatment of skin with the methods or compositions of the invention include, alone or in combination, the bicyclic aromatic compounds defined above, other compounds which have retinoid-type activity, free-radical scavengers, hydroxy or keto acids or derivatives thereof.

The term “free-radical scavenger” refers to, for example, alpha-tocopherol, superoxide dismutase. ubiquinol (e.g., coenzyme Q10) or certain metal-chelating agents. Hydroxy acids include, e.g., alpha-hydroxy acids such as lactic acid and glycolic acid or beta-hydroxy acids such as salicylic acid and salicylic acid derivatives such as the octanoyl derivative; other hydroxy acids and keto acids include malic, citric, mandelic, tartaric or glyceric acids or the salts, amides or esters thereof. Other anti-wrinkling agents and anti-skin aging agents useful in the invention include sulfur-containing D and L amino acids and their derivatives and salts, particularly the N-acetyl derivatives, a preferred example of which is N-acetyl-L-cysteine; thiols, e.g. ethane thiol; fat-soluble vitamins, ascorbyl palmitate, ceramides, pseudoceramides (e.g., pseudoceramides described in U.S. Pat. Nos. 5,198,210; 4,778,823; 4,985,547; 5,175,321, all of which are incorporated by reference herein), phospholipids (e.g., distearoyl lecithin phospholipid), fatty acids, fatty alcohols, cholesterol, plant sterols, phytic acid, lipoic acid; lysophosphatidic acid, and skin peel agents (e.g., phenol and the like), and mixtures thereof. Preferred fatty acids or alcohols are those that have straight or branched alkyl chains containing 12-20 carbon atoms. A particularly preferred fatty acid is linoleic acid since linoleic acid assists in the absorption of ultraviolet light and furthermore is a vital component of the natural skin lipids. Other non-limiting examples of suitable anti-wrinkle actives for use herein are described in U.S. Pat No. 6,217,888, which description is incorporated herein by reference.

Compositions for cosmetic treatment of skin may further include sunscreens to lower skin's exposure to harmful UV rays. Sunscreens include those materials commonly employed to absorb or block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, PARSOL MCX and BENZOPHENONE-3, respectively. Dermascreen may also be used.

Many other sunscreens are known to those of skill in the art. In some embodiments, sunscreens are FDA-approved or approved for use in the European Union. For example, FDA-approved sunscreens may be used, singly or, preferably, in combination. See, e.g., U.S. Pat. Nos. 5,169,624; 5,543,136; 5,849,273; 5,904,917; 6,224,852; 6,217,852; and Segarin et al., chapter Vil, pages 189 of Cosmetics Science and Technology, and Final Over-the-Counter Drug Products Monograph on Sunscreens (Federal Register, 1999:64:27666-27963), all of which are incorporated herein by reference. The exact amount of sunscreen employed in-the compositions can vary depending upon the degree of protection desired from the sun's UV radiation.

Cosmetic compositions of the invention may further include anti-acne agents. Antiacne agents include benzoyl peroxide, antibiotics, e.g., erythromycin, clindamycin phosphate, 5,7-dichloro-8-hydroxyquinoline, resorcinol, resorcinol acetate, salicylic acid, azaleic acid, long chain dicarboxylic acids, sulfur, zinc, retinoids, antiandrogens, and various natural agents such as those derived from green tea, tea tree oil, and mixtures thereof. Other non-limiting examples of suitable anti-acne agents for use herein are described in U.S. Pat. No. 5,607,980, which description is incorporated herein by reference.

Other cosmetic and dermatological agents include anticellulite agents. Anticellulite agents include isobutylmethylxanthine, caffeine, theophylline, theobromine, aminophylline, yohimbine, and mixtures thereof. Yet other cosmetic or dermatological agents that complement cosmetic treatment of skin include alpha-interferon, estradiol; progesterone; pregnanalone; methylsolanomethane (MSM); copper peptide (copper extract); plankton extract (phytosome); broparoestrol; estrone; adrostenedione; androstanediols; etc.

The compositions of the present invention may contain a wide range of additional components. The CTFA Cosmetic Ingredient Handbook, Seventh Edition, 1997 and the Eighth Edition, 2000, which are incorporated by reference herein in their entirety, describes a wide variety of ingredients commonly used in skin care compositions, which are suitable for use in the compositions of the present invention. Other topically-applied compounds are listed in Remington's Pharmaceutical Sciences, 20th Ed., Lippincott Williams & Witkins, Baltimore, Md. (2000) (hereinafter Remington's), U.S.

Pharmacopeia and National Formulary, The United States Pharmacopeial Convention, Inc., Rockville, Md. and Physician's Desk Reference, Medical Economics Co., Inc., Oradell, N.J. incorporated herein by reference. The concentration of the other active ingredient in formulations provided by the invention is that which provides an effective amount of the other active ingredient; these concentrations are well-known in the art.

In one embodiment of the invention, disclosed is a method of treating skin tissue in the face or selected areas of the body. The method comprises: (a) obtaining autogenous mesenchymal stem cells; (b) applying said autogenous mesenchymal stem cells topically onto the skin tissue; and, (c) rejuvenating, augmenting, and/or repairing the skin tissue. In one aspect, the method further comprises obtaining autogenous bone marrow concentrate or adipose-derived SVT containing said autogenous mesenchymal stem cells. In another aspect, the method further comprises aspirating autogenous bone marrow to obtain said autogenous bone marrow concentrate. Illustratively, aspiration of said autogenous bone marrow results in obtention of about 5 milliliters to about 55 milliliters of said autogenous bone marrow concentrate. In another aspect, the method further comprises aspirating autogenous bone marrow of an ilium of a pelvis. In another aspect, treatment of skin tissue with said autogenous mesenchymal stem cells comprises applying topically said autogenous mesenchymal stem cells onto said skin tissue. In an alternative variation, instead of obtaining and using autogenous bone marrow concentrate containing said autogenous mesenchymal stem cells, other suitable autogenous biologic products containing said autogenous mesenchymal stem cells may be obtained and used, such as autogenous biologic products obtained from peripheral blood, synovium, periosteum, skeletal muscle, adipose tissue, and other autogenous mesenchymal stem cells sources known to those skilled in the art.

In another embodiment of the invention, disclosed is a method for treating skin tissue by using autogenous mesenchymal stem cells. The method comprises: (a) obtaining autogenous mesenchymal stem cells; (b) applying said autogenous mesenchymal stem cells onto said skin tissue; (c) rejuvenating, augmenting, and/or repairing said skin tissue. In one aspect, the method further comprises obtaining autogenous bone marrow concentrate containing said autogenous mesenchymal stem cells. The method further comprises aspirating autogenous bone marrow or suctioning adipose tissue to obtain said autogenous bone marrow concentrate, or SVT. Illustratively, the method further comprises aspirating said autogenous bone marrow to obtain about 5 milliliters to about 55 milliliters of said autogenous bone marrow concentrate. The method further comprises aspirating autogenous bone marrow of an ilium of a pelvis. The method further comprises minimally manipulating the autogenous bone marrow by processing it as described in Pettine, K. A., U.S. Pat. No. 9,408,874 (Aug. 9, 2016), which is incorporated herein by reference in its entirety.

BMA (bone marrow aspirate) and BMC (bone marrow concentrate) are known to contain hematopoietic as well as MSC populations. The method of extraction is typically correlative to the source of these cells. Typically, the novel technology utilizes an approach, whereby approximately 10 cc of BMA is drawn with typically frequent rotation and repositioning. Additional draws are done after deeper placement of a needle in the iliac crest.

Bone marrow collection and processing to BMC is carried out herein as described in Pettine, K. A., U.S. Pat. No. 9,408,874 (Aug. 9, 2016), which is incorporated herein by reference in its entirety. This provides a pre-determined amount of a processed bone marrow concentrate with a pre-determined amount of a pre-mixture, where the pre-mixture includes quantities of anticoagulant solution, dextrose and phosphate buffered saline. Typically, the iliac crest, and more typically the posterior iliac crest, is where bone marrow aspirate may be harvested in a surgical setting, however any suitable area where BMA may be extracted may be used. The novel cellular “snapshot” is derived via timely methodologies specific to non-expanded, minimally manipulated, autologous cell and associated endogenous microenvironment (“milieu”). Typically, once extracted, the BMA and the resulting BMC is obtained by isolation of the desired cell populations via centrifugation. However, any suitable means may be used to obtain the BMC from the BMA. When centrifugation is used, the BMA is separated according to the slightly differing specific gravities of the aspirated cell types. The cells contained in the BMA can be stratified under centrifugation. The volume, rate, and time of centrifugation are important for controlling the resulting biologic factors contained within the endogenous milieu. Typically, the longer the processing time and/or the more agitation and handling, the lower the oxygen level in the extracted cells which typically include cellular constituents and components sequestered in the residual endogenous milieu, wherein the milieu typically includes antigens, surface biomarkers, proteins and growth factors for angiogenesis, osteogenesis, other regenerative outcomes, and the like. In some embodiments, the degradative manipulation and resultant influence during processing is limited. The resulting, unadulterated milieu may retain a large number of unchanged biologic drivers, markers and signals that are dose appropriate and specific to the cascade of healing found through the native physiology. The stratification and selection of MSCs and progenitors from this population may influence traits such as specific plasticity and immunomodulation. Typically, the time between extraction and re-implantation or re-injection is within 1 hour, and more typically within 30 minutes, and still more typically within 20 minutes. Thus, a point of care approach may be typically implemented with the novel technology. Optionally, once the BMA has been centrifuged, the resulting stratified cell layers may be prepared for delivery to the patient. Typically, the containers, anticoagulants used, and the delivery media used for interim storage and delivery are also prepared during this step. In some embodiments, preserving endogenous proteins, structure and morphology resides within this step, as too great a deviation from the oxygen, microenvironment and stress factors can lead to changes in the composition of the milieu. Typically, the proteins, structure, and morphology are not significantly altered. In one embodiment, the novel delivery media formulation is tailored to preserve the extracted cells and their endogenous factors, while maintaining cell health and identity. Typically, a premixture including an aqueous solution of anticoagulant (ACD-A), an equal amount of dextrose (50%), and phosphate buffered saline (PBS), or the like is pre-mixed and aliquoted in a volume to typically match or approximate the cellular matrix extracted from the centrifugation stratification layers at a ratio of about 1:1. More typically, the premixture is added to the cellular matrix with specific volumes being matched to, or slightly greater than 50/50 by volume, although the ratio may be greater, such as 2:1 or even higher. In some embodiments, the steps in extracting, isolating, separating, re-extracting, dosing, mixing, and delivery impact the cell population, endogenous proteins, surface structural and biomarkers, associated with the compositional regenerative capacity. Typically, the shorter the time consumed by the above-mentioned steps, the less adulterated the original milieu composition will become.

The skin-tissue treatment with MSCs disclosed herein may be used to repair, treat, or ameliorate various aesthetic or functional conditions (e.g., defects) of the skin tissue through augmentation. The MSCs of the present embodiments may provide an important resource for rebuilding or augmenting damaged or lost tissue. In addition, the MSCs may be used for augmenting soft tissue not associated with injury by adding bulk to a soft tissue area, opening, depression, or void in the absence of disease or trauma, such as for “smoothing”.

### EXAMPLES

The following examples further illustrate specific embodiments of the invention. However, the following examples should not be interpreted in any way to limit the invention. Thus, it is understood that modifications which do not substantially affect the activity of the various embodiments of this invention are also provided within the definition of the invention provided herein. Accordingly, the following examples are intended to illustrate but not limit the present invention.

### Example 1

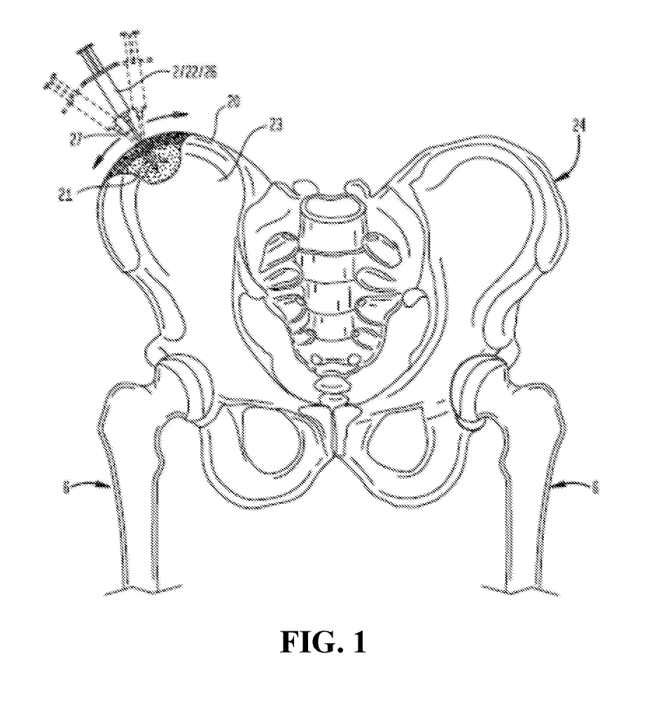

Obtention of Autogenous MSCs: Referring to FIG. 1, autogenous MSCs (2) are multipotent stromal cells that can differentiate into a variety of cell types, including: osteoblasts (bone cells), chondrocytes (cartilage cells), myocytes (muscle cells) and adipocytes (fat cells). Autogenous MSCs (2), suitable for use in embodiments of the inventive method, are typically obtained from bone marrow (21) or fractions thereof; however, this is not intended to limit obtention of autogenous MSCs (2) suitable for use with the inventive method solely from bone marrow (21) and it is understood that autogenous MSCs (2) may be isolated from other tissues such as: peripheral blood, synovium, periosteum, skeletal muscle, or adipose tissue. Additionally, as to certain embodiments, it is understood that autogenous MSCs (2), whether from bone marrow (21) (or fractions thereof) or other tissue sources, can optionally be expanded in media to obtain an amount of autogenous MSCs (2) for use in embodiments of the inventive method.

Typically, bone marrow aspirates can have a cellularity of marrow (21) of about 15 to about 30 million mononuclear cells per milliliter (“MNC/mL”), whereas less than 10 million MNC/mL usually means a rather diluted sample provided the donor was healthy and the marrow not fibrotic. The average colony forming unit-fibroblasts (“CFU-f”) content of healthy human bone marrow is about 100 CFU-f/million MNC. Accordingly, bone marrow concentrate (22) (“BMC”) resulting from aspirating bone marrow (21) can contain a few thousands (about 1500 to about 3000) CFU-f/mL of bone marrow (21).

As to particular embodiments, autogenous MSCs (2) obtained from bone marrow (21) or adipose tissue may be utilized in embodiments of the inventive method as autogenous BMC (22) or adipose-derived stromal vascular fraction (SVF) without further processing; although autogenous MSCs (2) can be contained or entrained in other biocompatible materials or compositions known in the relevant art. One preferred source of autogenous BMC (22) can be the bone marrow (21) of the ilium (23) of the greater pelvis (24); although autogenous BMC (22) obtained from other sites may be utilized. Adipose tissue is typically suctioned from the abdomen but other anatomical locations can also be used. Adipose-derived SVF, as has been described in the art, may be used; for example, as described by Oberbauer, E., et al., “Enzymatic and Non-Enzymatic Isolation Systems for Adipose Tissue-Derived Cells: Current State of the Art,” Cell Regeneration, 215, 4:7, the disclosure of which is incorporated herein by reference in its entirety.

### Example 2

Bone Marrow Collection and Processing: Following is an illustrative example of bone marrow collection and processing to BMC, which was carried out as described in Pettine, K. A., U.S. Pat. No. 9,408,874 (Aug. 9, 2016), which is incorporated herein by reference in its entirety. Accordingly, 60 cc of bone marrow aspirate was collected over ACD-A as needed per process. The marrow was processed using the bone marrow concentration system according to the detailed protocol. The patient was given IV antibiotics and placed prone on an image table. Intravenous Versed and Fentanyl was administered and the skin was anesthetized with buffered 1% Lidocaine. The aspirator was rinsed and the syringes were transferred with heparin solution, approximately 1000 U/ml. The heparin solution coated the inner surface of the 60-cc aspiration needle and trephine needle. The remaining heparin was expelled from the syringe. 6 cc of ACD-A was aspirated into the 60-cc syringe. Bone marrow was aspirated from the posterior iliac crest when the patient was positioned prone on a fluoro table. The right iliac wing was prepped and draped according to standard surgical protocols. A trephine needle and 60 cc syringe was used to remove the marrow. The surgeon inserted the trephine needle percutaneously through the skin until the bony surface of the iliac crest was felt. Using a mallet, the needle was then inserted to a depth of 3-4 cm into the crest. This was accomplished with fluoroscopic guidance. A 60-cc syringe containing 6 ml of acid citrate dextrose anticoagulant solution (ACD-A) (10% of the final volume) was attached to the needle. The marrow was aspirated by pulling the plunger back and allowing the syringe to fill to the 10-cc level. The needle was repositioned by advancing 1.5-2 cm and an additional 10 cc of aspirate was obtained. This process was repeated until 60 cc of iliac aspirate was obtained. Once the final marrow volume was reached, the solution was mixed by gentle rocking of the syringe as the syringe was rotated on its long axis. The marrow was then ready for processing. The marrow was mixed with anticoagulant solution by gently turning the syringe after each 10 cc of aspirate collection. The extracted marrow was placed in an isolating canister and loaded into the centrifuge. The marrow was centrifuged for about 12 minutes at about 3200 rpm. The processed marrow was drawn with a syringe from the centrifuge and then rocked while rotating the syringe on its long axis. The syringe was then presented to a sterile field. The amount of bone marrow concentrate removed from the centrifuge equaled the amount to be used. The cell delivery media was pre-mixed and aliquoted to 1 cc, composing of 0.5 cc of ACD-A and 0.5 cc of dextrose (50%). The delivery media was injected into a closed vial containing the cell components slowly, in order to homogenize the mixture and incorporate oxygen and turbidity mixing in a closed, sterile system. Optionally, one or more fillers of the types used in the art may be added, but are not required. The resulting mixture was then used for treatment of skin tissue without delay. Typically, the entire procedure from beginning of BMA collection to treatment of a patient's skin tissue is performed in less than one hour.

Following is a second illustrative example of bone marrow collection and processing to BMC. Referring to FIG. 1, about 55 milliliters (“mL”) of autogenous bone marrow aspirate (26) (“BMA”) was collected over about 5 mL acid citrate dextrose-anticoagulant (“ACD-A”) from the participant's posterior iliac crest (20) of the ileum (23) of the greater pelvis (24). The procedure was performed with intravenous sedation consisting of VERSED® (also known as Midazolam) (8-chloro-6-(2-fluorophenyl)-1-methyl-4H-imidazo[1,5-a]-[1,4]benzodiazepine, CAS Number: 59467-70-8) and FENTANYL® (N-(1-(2-phenylethyl)-4-piperidinyl)-N-phenylpropanamide, CAS Number 437-38-7). Positioning of a JAMSHIDI® bone marrow biopsy needle (27) in the iliac crest (20) was confirmed by fluoroscopy. BMA (26) was collected in a 60 mL syringe in a series of discrete pulls on the plunger (targeting a collection of 5-10 mL per pull), with repositioning of the needle tip between pulls based on the reported enrichment of autogenous MSCs (20) as described by Hernigou, et al., J Bone Joint Surg Br 2005 87 (7) 896-902. The BMA (26) was captured using a ART21® BMC cell capturing device available from Celling Biosciences, Austin, Tex. to obtain BMC (22). Typically, a BMC (22) volume of about 12 mL was drawn from the ART21® BMC cell capturing device.

### Example 3

Technique for Treatment of Skin Tissue. The following is a technique used to treat skin tissue in accordance with the invention, resulting in rejuvenation, augmentation, and/or repairing of the skin tissue. While in this example the use of autogenous BMC as obtained above is described, it is understood that any suitable biologic product containing autogenous or allogeneic mesenchymal stem cell can be utilized. The technique involves the following illustrative procedure. The BMC is formulated in a cosmeceutical facial cream, lotion, and/or serum for topical application, with or without additional growth factors, exosomes, peptides, and/or other proteins and biologically active substances, including, but not limited to, those discussed herein. Typical skin cream formulations may include one of more of the following general types of ingredients: Emollients, in the form of plant oils, mineral oils, shea butter, cocoa butter, petrolatum, cholesterol, silicones or animal oils (including emu, mink and lanolin). These lubricating ingredients soften and smooth skin while helping it to retain moisture. In some embodiments, jojoba, squalene and lanolin represent recited emollients because they bear the greatest similarity to sebum (the skin's natural moisturizing agent), are the least comedogenic (pore-clogging), and are most compatible with the skin's biochemistry. Thickening agents like triglycerides, palmitates, myristates and stearates are waxier, but necessary for the fundamental base and texture of a moisturizing formulation. Water-binding agents are ingredients that keep water in the skin. Humectants (including sorbitol, glycols, glycerins and sodium PCA), which attract water to skin, may be desirable in formulations designed to treat/prevent skin damaged by sun and dehydration, but they are less useful in promoting water retention by the skin. Soothing agents and anti-irritants, such as bisabolol, allantoin, burdock root, aloe, licorice root, glycyrrhetinic acid, green tea and chamomile extract, may be added to help skin handle ingredients that may cause irritation. Vitamins and antioxidants, including vitamins A, C and E, may be used to promote cell turnover, healing and dehydration. Alpha hydroxy acids (AHAs) and beta hydroxy acids (BHAs) have been shown to clear pores and remove dead skin, resulting in smoother, moister skin. AHA formulations include glycolic acid and lactic acid, while the use fruit or citrus acid, sugarcane, or even sour milk may be substituted. One BHA ingredient is salicylic acid. However, high levels of AHAs may feel tingly on certain skin types. Also, because AHA increases sun sensitivity, sun protection (e.g., addition of physical and/or chemical sunscreen agents) may be desirable for formulations which incorporate an AHA.

In one embodiment, the formulated skin cream may combine therapeutically effective amounts of BMC (or concentrates or extracts thereof) with a thickener, a humectant, allantoin, purified water, and at least one preservative.

In another embodiment, the thickener may comprise a combination of polyethylene glycol (PEG), a vegetable-based fatty alcohol(s), and a copolymer(s).

Some preferred vegetable-based fatty alcohols may include, but are not limited to: decyl alcohol, octyl-decyl alcohol, lauryl alcohol, lauryl-myristyl alcohol, myristyl alcohol, ceto-stearyl alcohol and its various blends, cetyl alcohol, and stearyl alcohol.

Copolymers may include those conventionally used in cosmeceuticals, as known by those skilled in the art.

In another embodiment, the thickener may comprise PEG-150, decyl alcohol, and SMDI copolymer.

Some humectants may include, but are not limited to: sodium PCA, glycerine, propylene glycol, sorbitol, hyaluronic acid, urea, and lactic acid.

Some preservatives may include, but are not limited to: heterocyclic compounds, methylparaben, propylparaben, diazolidinyl urea, phenoxyethanol, DMDM hydantoin, sorbic acid, benzyl alcohol, formaldehyde, triclosan and EDTA.

Some heterocyclic compounds may include, but are not limited to: methylisothiazolinone, methylchloroisothiazolinone, and caffeine.

In one embodiment, the formulated cream may combine the BMA (or concentrates or extracts thereof) with PEG-150/decyl alcohol/SMDI copolymer, sodium PCA, allantoin, purified water, methylisothiazolinone, and methylparaben.

In other embodiments, the BMA may be formulated into pharmaceuticals in the form of skin patches, injectables, hydrogels and into any other appropriate formulation known to one of skill in the art.

The pharmaceutical/cosmeceutical formulations may be delivered to a subject via a variety of routes using standard procedures well known to those of skill in the art. For example, such delivery may be site-specific or general topical administration, including the use of transdermal stamps. Also, they may be formulated to function as controlled, slow release vehicles. In addition, specifically designed disposable applicators may also be used (e.g., for eyelash rejuvenation/growth).

It is to be understood that, as contemplated herein, in any and all instances throughout this application where Applicants refer to treatment(s) using BMC and/or MSC, any and all of the various BMC and/or MSC delivery media or mixtures described herein may be used, whether the specific details of the BMC and/or MSC delivery media or mixture are recited or not.

### Example 4

Expanding Human MSCs (hMSCs). An embodiment of the disclosed invention provides MSC preparations and/or formulations, and associated methods, for treating skin tissue using autogenous or allogeneic MSCs, wherein the MSCs have been cultured and/or culture-expanded. Evidence exists that this may enhance the efficacy as well as safety of hMSC therapeutics. Various techniques and methods for expanding the MSCs are well known and have proliferated in the art, including all aspects of preparing the culture media, cell bioprocessing protocols, and related steps, while maintaining the therapeutic and differentiation capacity of the MSCs. For the purpose of this invention, the following publications encompass the techniques and methods for expanding the MSCs of the invention prior to their use in treatment of skin tissue. Thus, the disclosures of the following publications are hereby incorporated by reference in their entirety:
Jung, S., et al., “Ex Vivo Expansion of Human Mesenchymal Stem Cells in Defined Serum-Free Media,” Stem Cells International, Volume 2012, Article ID 123030, pages 1-21.
Bruedigam, C., et al., “Basic Techniques in Human Mesenchymal Stem Cell Cultures: Differentiation into Osteogenic and Adipogenic Lineages, Genetic Perturbations, and Phenotypic Analyses,” Curr. Protoc. Stem Cell Biol. 17:1H.3.1-1H.3.20.
Battula, V. L., et al., “Human placenta and bone marrow derived MSC cultured in serum-free, b-FGF-containing medium express cell surface frizzled-9 and SSEA-4 and give rise to multilineage differentiation,” Differentiation (2007) 75:279-291.
Ikebe, C., et al., “Mesenchymal Stem Cells for Regenerative Therapy: Optimization of Cell Preparation Protocols” BioMed Research International, Volume 2014, Article ID 951512, pages 1-11.

### Example 5

Obtaining Allogeneic-Derived MSCs. An embodiment of the disclosed invention provides MSC preparations and/or formulations, and associated methods, for treating skin tissue using either autogenous or allogeneic MSCs. Regarding allogeneic MSCs, methods and sources of obtention have proliferated in the art in recent years. Illustratively, the allogeneic MSCs may be obtained from the iliac wing, the removed bone from a total hip or knee replacement, or from other appropriate aspiration sites from one or more screened donors. For the purpose of this invention, the foregoing publications in Example 4 above by Jung et al., Bruedigam et al., Battula et al., and Ikebe et al. encompass the techniques and methods for obtaining the allogeneic MSCs of the invention; and, accordingly, the disclosures of these four publications are hereby incorporated by reference in their entirety for that purpose. Additionally, various companies have emerged that provide specialized culture media to produce MSCs that have the ability to maximize fibroblast differentiation and increase collagen fiber production, elastin fiber production, and glycosaminoglycan production, as well as to maximize production of various growth factors, including bFGF, KGF-2, IGF-1, EGF, and SOD-1. For the purpose of this invention, the following is an illustrative list of companies that provide specialized culture media to produce the allogeneic MSCs of the invention: PromoCell, Life Cell Technology, StemPro®, StemMACS™, and Cell Applications Inc.

### Example 6

Modification of Expanded MSC Growth Media by Exposure to Pathologic Tissue and/or Known Cytokines. An embodiment of the disclosed invention provides MSC preparations and/or formulations, and associated methods, for treating skin tissue, wherein the expanded MSC growth media are modified to prepare the MSCs being expanded to treat underlying conditions by exposing them to the pathologic tissue or known cytokines. This is believed to be important since, unlike the unexpanded autogenous BMAC, they will be deactivated/lysed prior to being topically applied to the skin tissue. In this regard, methods for modification of expanded MSC growth media by exposing them to the pathologic tissue or known cytokines have proliferated in the art in recent years. For the purpose of this invention, the foregoing publications in Examples 4 and 5 above by Jung et al., Bruedigam et al., Battula et al., and Ikebe et al. encompass the techniques and methods for modifying the expanded MSC growth media by exposure to pathologic tissue or known cytokines; and, accordingly, the disclosures of these four publications are hereby incorporated by reference in their entirety for that purpose.

### Example 7

Inclusion of Additives to Induce Keratinocyte Proliferation and Collagen Production. An embodiment of the disclosed invention provides MSC preparations and/or formulations, and associated methods, for treating skin tissue, wherein the MSC culture media are supplemented with additives known to induce keratinocyte proliferation and collagen production. This is believed to be important because keratinocyte proliferation and collagen production are known to decrease with aging. Methods for inclusion of various additives that induce keratinocyte proliferation and collagen production have proliferated in the art in recent years. For the purpose of this invention, the foregoing publications in Examples 4-6 above by Jung et al., Bruedigam et al., Battula et al., and Ikebe et al. encompass additives, techniques and methods for inducing keratinocyte proliferation and collagen production; and, accordingly, the disclosures of these four publications are hereby incorporated by reference in their entirety for that purpose.

### Example 8

Post-Treatment of MSC-Treated Skin Tissue. An embodiment of the disclosed invention provides MSC preparations and/or formulations, and associated methods, for treating skin tissue, wherein after treatment of the skin tissue, one or more post-treatments of the skin tissue are undertaken to enhance the MSC function after application. It is well known in the art that optimization of penetration and/or delivery through skin tissue can significantly improve the overall outcome of the treatment, and a wealth of agents, methods and techniques have been developed for that purpose. Post-treatments have been reported to be effective in accomplishing as much as a 10-fold enhancement relative to procedures that do not employ post-treatment methods. Optimization of penetration and/or delivery can be accomplished, illustratively, by the use of plastic covers and/or masks, and the like; by modifications to the stratum corneum, e.g., by use of hydration agents and chemical enhancers; by ozonation; by ablation and follicular delivery; and/or by electrically assisted methods (e.g., ultrasound, iontophoresis, electroporation, magnetophoresis, laser light and photomechanical waves). Better optimization can be accomplished by synergistic use of a multiplicity of the foregoing methods. For the purpose of this invention, as contemplated herein, the following publications encompass the techniques and methods for post-treatment of MSC-treated skin tissue. Thus, the disclosures of the following publications are hereby incorporated by reference in their entirety:
Barry, B. W., “Novel Mechanisms and Devices to Enable Successful Transdermal Drug Delivery,” European Journal of Pharmaceutical Sciences, 14, (2001), 101-114.
Santus, G. C., et al., “Transdermal Enhancer Patent Literature,” Journal of Controlled Release, 25, (1993), 1-20.
Lane, M. E., “Skin Penetration Enhancers,” International Journal of Pharmaceutics, 447 (2013), 12-21.
Williams, A. C., et al., “Penetration Enhancers,” Advanced Drug Delivery Reviews, 64, (2012), 128-137.
Zhai, H., et al., “Occlusion vs. Skin Barrier Function,” Skin Research and Technology, 8, (2002), 1-6.

As can be easily understood from the foregoing, the basic concepts of the present invention may be embodied in a variety of ways. The invention involves numerous and varied embodiments of a treatment of skin tissue with autogenous and/or allogeneic mesenchymal stem cells resulting in improvement, restoration, rejuvenation, augmentation, and/or repair of said skin tissue towards normal physiologic structure.

As such, the particular embodiments or elements of the invention disclosed by the description or shown in the figures or tables accompanying this application are intended to be exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “treatment” should be understood to encompass disclosure of the act of a “treating”, whether explicitly discussed or not, and, conversely, were there effectively disclosure of the act of “treating”, such a disclosure should be understood to encompass disclosure of a “treatment” and even a “means for treating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition each definition hereby incorporated by reference.

All numeric values herein are assumed to be modified by the term “about”, whether or not explicitly indicated. For the purposes of the present invention ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes, for example, the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

It is to be understood that, as used herein, the grammatical conjunction “and/or” refers throughout to either or both of the stated possibilities.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) a treatment of skin tissue with autogenous mesenchymal stem cells herein disclosed and described, ii) the related methods disclosed and described, iii) similar equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following claims are desired to be protected. It will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. In addition, all publications cited herein are indicative of the level of skill in the art and are hereby incorporated by reference in their entirety as if each had been individually incorporated by reference and fully set forth.

What is claimed is:

1. A cosmetic or dermatological formulation for rejuvenating, augmenting, and/or repairing skin tissue of a human subject, said formulation comprising autogenous or allogeneic mesenchymal stem cells.

2. The formulation of claim 1 further comprising autogenous or allogeneic bone marrow concentrate or adipose-derived stromal vascular fraction that contains said mesenchymal stem cells.

3. The formulation of claim 2, wherein said autogenous bone marrow concentrate is from an ilium of the pelvis and said adipose-derived stromal vascular fraction is from the abdomen or another appropriate anatomical location of said human subject or a donor.

4. The formulation of claim 3, wherein the autogenous bone marrow concentrate or adipose-derived stromal vascular fraction is minimally manipulated.

5. The formulation of claim 1, wherein the autogenous or allogeneic mesenchymal stem cells have been culture expanded.

6. The formulation of claim 1 further including a cosmetic base having from about 0.01% to 10% w/w of a mesenchymal stem cells preparation.

7. The formulation of claim 1 further comprising a tissue growth factor.

8. The formulation of claim 1 further comprising delivery systems including at least one of liposomes, cyclodextrins, polymer systems, or hyaluronic acid.

9. The formulation of claim 1 further including one or more of the following: lotions, creams, oils, gels, hydrogels, powders, serums, salves, foundations, facial masks, lip care products, sunscreens, hair care products, conditioners, hair care treatments, hot oil treatments, skin cleansers, anti-acne agents, exfoliants, ointments, and mixtures thereof.

10. The formulation of claim 1 further comprising one or more penetration enhancers and/or penetration techniques, including one or more of the following: amphiphiles, anionic surfactants, cationic surfactants, amphoteric surfactants, nonionic surfactants, fatty acids, alcohols, various iontophoresis techniques, and mixtures or combinations thereof.

11. The formulation of claim 1 further comprising one or more anticellulite agents, including one or more of the following: isobutylmethylxanthine, caffeine, theophylline, theobromine, aminophylline, yohimbine, and mixtures thereof.

12. The formulation of claim 1 further including one or more cosmetically-acceptable, cosmeceutically-acceptable, or pharmaceutically-acceptable preparation.

13. The formulation of claim 1 further including one or more of the following: one or more solvents, one or more botanicals, and one or more emollients.

14. A method of treatment of one or more conditions of the skin tissue of a human subject including wrinkling, folds, sagging, age spots, uneven pigmentation, thinning, elasticity, scarring, surface roughness, surface vessels, redness, pore size, and burns, said method comprising topical application to the skin tissue of the subject having one or more of the aforesaid conditions an effective amount of the formulation of claim 1, resulting in rejuvenation, augmentation, and/or repair of the skin tissue.

15. The method of treatment of claim 14, wherein the formulation further comprises autogenous or allogeneic bone marrow concentrate or adipose-derived stromal vascular fraction that contains said mesenchymal stem cells; wherein said mesenchymal stem cells may be culture expanded to produce pure mesenchymal stem cells.

16. The method of treatment of claim 14, wherein the effective amount of the formulation includes a cosmetic base having from about 0.01% to 10% w/w of a mesenchymal stem cells preparation.

17. The method of claim 14, wherein the topical application is administered in more than one treatment session.

18. The method of claim 14, further comprising inducing tissue growth of the skin of a burn victim.

19. A cosmetic or dermatological kit comprising the formulation of claim 1.

20. The kit of claim 19, further comprising: a) a container; b) a label; c) an optional applicator; and d) instructions which provide methods of applying the cosmetic or dermatological formulation.
